(19) United States
(12) Patent Application Publication
(10) Pub. No.: US 2011/0054459 A1
(43) Pub. Date: Mar. 3, 2011

(54) Ecogenic Cooled Microwave Ablation Antenna

(75) Inventor: Darion Peterson, Bouler, CO (US)

(73) Assignee: Vivant Medical, Inc.

(21) Appl. No.: 12/548,644

(22) Filed: Aug. 27, 2009

(51) Int. Cl. A61B 18/18

(52) U.S. Cl. 606/33

(57) ABSTRACT

An electrosurgical positioning and energy delivery system includes a positioning introducer, a microwave energy delivery device and a jacket configured to slideably receive one of the positioning introducer and the microwave energy delivery device. The positioning introducer and the jacket form a positioning assembly and are configured for percutaneous insertion in patient tissue. The positioning assembly is visible percutaneously to an imaging system. The microwave energy delivery device and the jacket form a microwave energy delivery assembly. The microwave energy delivery assembly is configured to circulate cooling fluid therethrough during delivery of microwave energy to the patient tissue.

### BACKGROUND

1. Technical Field

The present disclosure relates generally to medical/surgical ablation assemblies and methods of their use. More particularly, the present disclosure relates to an ecogenic cooled microwave ablation system and antenna assemblies configured for direct insertion into tissue for diagnosis and treatment of the tissue and methods of using the same.

2. Background of Related Art

In the treatment of diseases such as cancer, certain types of cancer cells have been found to denature at elevated temperatures (which are slightly lower than temperatures normally injurious to healthy cells). These types of treatments, known generally as hyperthermia therapy, typically utilize electromagnetic radiation to heat diseased cells to temperatures above 41° C. while maintaining adjacent healthy cells at lower temperatures where irreversible cell destruction will not occur. Other procedures utilizing electromagnetic radiation to heat tissue also include ablation and coagulation of the tissue. Such microwave ablation procedures, e.g., such as those performed for menorrhagia, are typically done to ablate and coagulate the targeted tissue to denature or kill it. Many procedures and types of devices utilizing electromagnetic radiation therapy are known in the art. Such microwave therapy is typically used in the treatment of tissue and organs such as the prostate, heart, and liver.

One procedure generally involves the treatment of tissue (e.g., a tumor) underlying the skin via the use of a percutaneously inserted microwave energy delivery device. The microwave energy delivery device penetrates the skin and is positioned relative to the target tissue, however, the effectiveness of such a procedure is often determined by the precision in which the microwave energy delivery device is positioned. Thus, the placement of the microwave energy delivery device requires a great deal of control.

### SUMMARY

The present disclosure describes an electrosurgical positioning and energy delivery system for direct insertion into tissue. The electrosurgical positioning and energy delivery system includes a positioning introducer, a microwave energy delivery device and a jacket configured to slideably receive one of the positioning introducer and the microwave energy delivery device. The positioning introducer and the jacket form a positioning assembly and are configured for percutaneous insertion in patient tissue. The positioning assembly is visible percutaneously to an imaging system. The microwave energy delivery device and the jacket form a microwave energy delivery assembly. The microwave energy delivery assembly is configured to circulate cooling fluid therethrough during delivery of microwave energy to the patient tissue.

In one embodiment the positioning introducer is hyperechoic. In another embodiment, the positioning introducer is visible to an ultrasonic imaging system and/or an MRI imaging system. The positioning introducer may include a treatment configured to improve visibility of the positioning introducer by an ultrasonic imaging system. The treatment may include a surface dispersion treatment, a dimpled surface and a surface of imbedded particles. The positioning introducer may include a resonant material that resonates when exposed to energy transmitted from the ultrasonic imaging system. One resonate material is a crystalline polymer.

In yet another embodiment, the positioning introducer includes a geometry that resonates at the frequency of the energy transmitted from the ultrasonic imaging system. The geometry is defined by at least one of wall thickness of the positioning introducer, a gap defined in a periphery of the positioning introducer, a series of grooves defined in a periphery of the positioning introducer and a fin extending from a periphery of the positioning introducer.

In yet another embodiment, the positioning introducer includes a non-ferromagnetic material that is percutaneously visible to an MRI imaging system. The non-ferromagnetic material may include one of a ceramic, titanium and plastic.

In yet another embodiment, the jacket, assembled with the positioning introducer, includes a geometry at a distal end thereof to facilitate tissue penetration.

In still yet another embodiment the microwave energy delivery assembly is adapted to connect to a microwave energy source that supplies a microwave energy signal. The microwave energy delivery may also be adapted to connect to a cooling fluid source that supplies cooling fluid.

A method for deploying an electrosurgical energy apparatus includes the steps of: providing an electrosurgical positioning and energy delivery system including a positioning introducer, a microwave energy delivery device and a jacket configured to slideably receive one of the positioning introducer and the microwave energy delivery device; forming a positioning assembly by slideably receiving the positioning introducer within the jacket; advancing the positioning assembly to target tissue whereby the advancement of the positioning assembly is percutaneously observed on a image system, the positioning assembly defining a pathway during tissue penetration; withdrawing the positioning introducer from the jacket, with the jacket remaining in situ; forming a microwave energy delivery assembly by slideably receiving the microwave energy delivery device within the jacket; treating target tissue with electrosurgical microwave energy; and withdrawing the microwave energy delivery assembly from the pathway.

The method may further include the steps of: connecting a fluid supply to the microwave energy delivery device and a cooling fluid return to the jacket and circulating the cooling fluid through at least a portion of the microwave energy delivery assembly to absorb thermal energy therefrom.

### BRIEF DESCRIPTION OF THE DRAWINGS

FIGS. 1A-1B are perspective views of the positioning assembly according to an embodiment of the present disclosure including a positioning introducer and an outer jacket;

FIG. 2A is an illustration of the positioning assembly of FIG. 1B partially inserted into tissue;

FIG. 2B is an illustration of the positioning introducer removed from the jacket after the jacket is positioned in a target tissue.

FIG. 3A is a perspective view of a microwave energy delivery assembly according to another embodiment of the present disclosure including a microwave energy delivery device and an outer jacket;

FIG. 3B is a cross sectional view of the assembled microwave energy delivery assembly of FIG. 3A;

FIG. 4A is an illustration of the microwave energy delivery device being inserted into the jacket positioned in a tissue pathway;

FIG. 4B is an illustration of the energy delivery device assembly positioned in a tissue pathway; and

FIGS. 5A-5D are prospective views of various jacket configurations according to an embodiment of the present disclosure.

### DETAILED DESCRIPTION

Embodiments of the presently disclosed assemblies, systems and methods are described in detail with reference to the drawing figures wherein like reference numerals identify similar or identical elements. As used herein and as is traditional, the term “distal” refers to the portion which is furthest from the user and the term “proximal” refers to the portion that is closest to the user. In addition, terms such as “above”, “below”, “forward”, “rearward”, etc. refer to the orientation of the figures or the direction of components and are simply used for convenience of description.

During invasive treatment of diseased areas of tissue in a patient, the insertion and placement of an electrosurgical energy delivery apparatus, such as a microwave antenna assembly, relative to the diseased area of tissue is important for successful treatment. Generally, assemblies described herein allow for placement of a microwave antenna in a target tissue in a two step process. In a first step, a positioning assembly is directly inserted and positioned into target tissue and in a second step the positioning introducer is removed from a positioning jacket and replaced with a microwave energy delivery device, the jacket and microwave energy delivery device thereby forming an energy delivery device assembly in the target tissue.

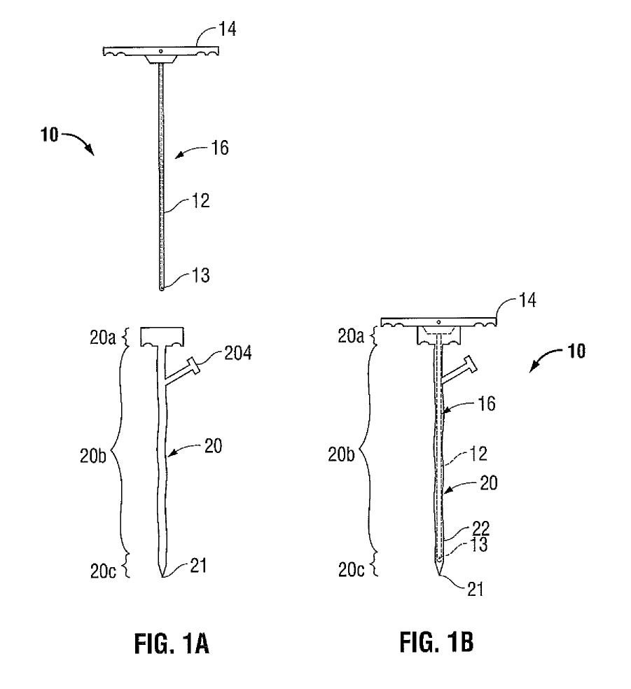

Referring now to FIGS. 1A-1B, a positioning assembly, according to an embodiment of the present disclosure, is shown as 10. The positioning assembly 10 includes a positioning introducer 16 and a jacket 20. The positioning introducer 16 includes a handle 14 that connects to an elongated shaft 12. The elongated shaft 12 includes a tip 13 at a distal end thereof. Jacket 20 includes a receiver portion 20a, a sheath portion 20b, a receptacle tip portion 20c and a fluid outlet 204. Sharpened tip 21 (on the distal end of the receiver portion 20a) is configured to be percutaneously inserted into tissue to define a pathway therethrough.

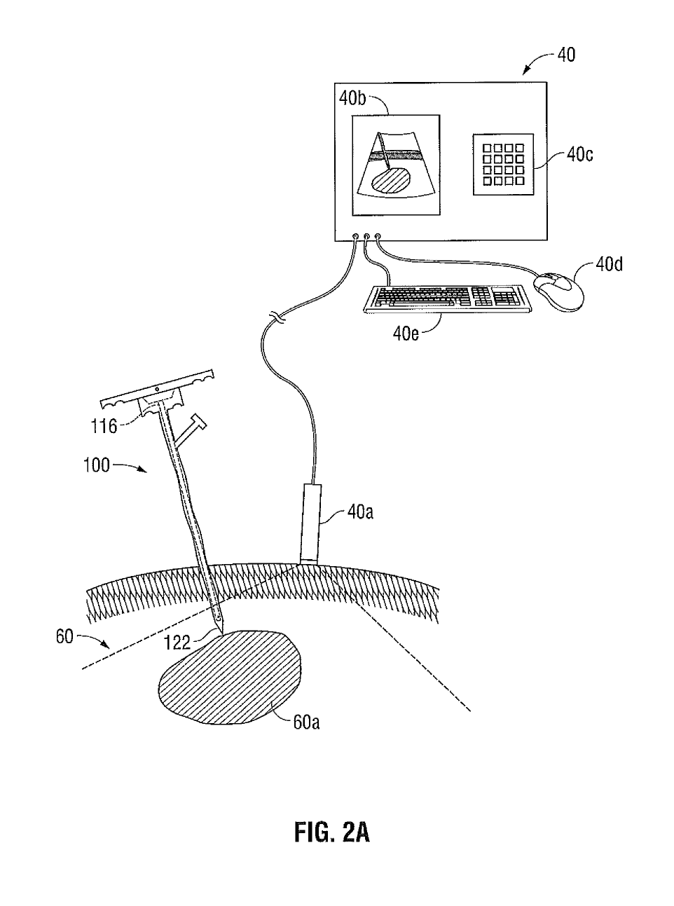

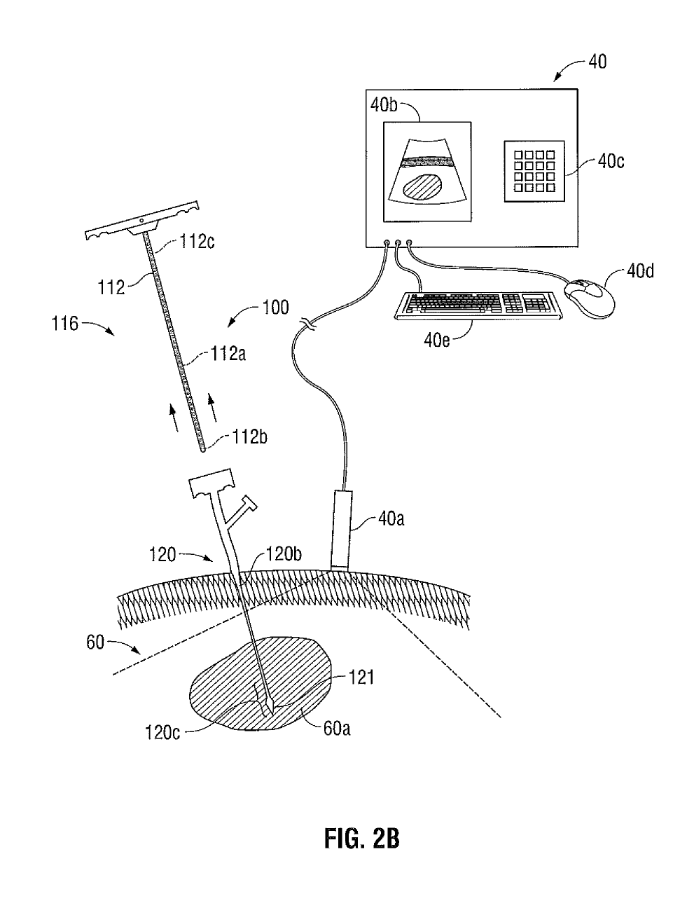

As illustrated in FIG. 2B, the positioning introducer 16 is configured to slideably engage the jacket 20 and forms a percutaneously insertable positioning assembly 10. Receiver portion 20a of the jacket 20 is configured to receive at least a portion of the handle 14 of the positioning introducer 16 thereby forming an assembly handle 15. Assembly handle 15, when grasped by a clinician, enables the clinician to control the positioning assembly 10 during insertion. Sheath portion 20b is configured to slideably engage the elongated shaft 12.

Receptacle tip portion 20c is configured to receive and engage at least a portion of tip 13 thereby forming a structurally rigid tip assembly 22 with the sharpened tip 21 on the distal end of the positioning assembly 10.

Elongated shaft 12 and tip 13 of positioning introducer 16 are configured to produce a highly identifiable image on a suitable imaging system used to aid in the positioning of an ablation device in target tissue. The elongated shaft 12 and tip 13 may be highly identifiable due to one or more materials used in their construction and/or one or more identifiable features incorporated into the design and/or the materials of the positioning introducer 16.

In one embodiment, the elongated shaft 12 and tip 13 of the positioning introducer 16 are readily identifiable by an ultrasonic imaging system 40, as illustrated in FIG. 2A. Ultrasonic imaging system 40 includes an imaging device 40a, such as, for example, a suitable ultrasonic transducer, a display 40b and one or more suitable input devices such as, for example, a keypad 40c, keyboard 40e, a pointing device 40d and/or an external display (not explicitly shown).

As illustrated in FIG. 2A, the positioning assembly 100 is percutaneously inserted into patient tissue 60. During insertion, the disposition of the positioning assembly 100 with respect to the target tissue is percutaneously observed on the display 40b of the imaging device 40. The hyperechoic positioning introducer 116 of the positioning assembly 100 is easily identifiable on display 40b. The positioning assembly 100 is guided by a clinician into a desirable position within a portion of the target tissue 60a while the clinician percutaneously observes the advancement of the positioning assembly 100 on the display 40b forming a pathway in tissue.

Various echogenic treatments may be applied to the positioning introducer 116 to enhance the ability of the ultrasonic imaging device 40 to replicate the positioning introducer on the display 40b. In one embodiment, the positioning introducer 116 includes a surface dispersion treatment. The surface dispersion treatment may include a dimpled surface or a surface imbedded with particles wherein the surface dispersion treatment creates wide angles of dispersion of the energy transmitted from the imaging device 40a. In another embodiment, the positioning introducer 116 is formed from a composite material that includes particles or fibers bonded within the structure wherein the orientation of the particles or fibers create a wider angle of dispersion of the energy transmitted from the imaging device 40a.

In yet another embodiment, the positioning introducer 116 includes resonant materials or structures configured to resonate when exposed to energy transmitted from the imagine device 40a. The positioning introducer 116 may include materials, such as crystalline polymers, that absorb energy and resonate when exposed to the energy transmitted from the imaging device 40a. Alternatively, the surface of the positioning introducer 116 may include specific geometries, such as, for example, wall thickness of the positioning introducer 116, gaps defined in a periphery of the positioning introducer 116, a groove or a series of groves defined in a periphery of the positioning introducer 116 and/or fins extending from a periphery of the positioning introducer 116, wherein the specific geometry is configured to resonate at the frequency of the energy transmitted from the imagine device 40a.

In yet another embodiment, a clinician may utilize a Magnetic Resonance Imaging (MRI) device to observe the positioning introducer 116 during the positioning step. The positioning introducer 116, when used with an MRI device, may include one or more non-ferromagnetic materials with very low electrical conductivity, such as, for example, ceramic, titanium and plastic.

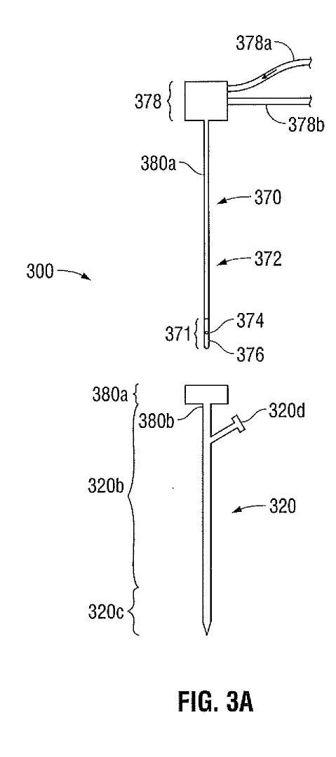

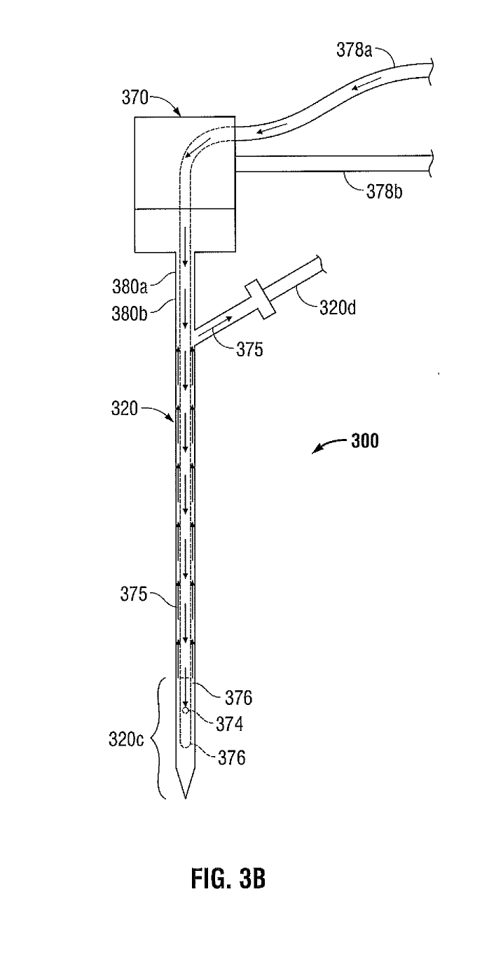

As illustrated in FIG. 2B, after positioning, where the positioning assembly 100 is properly positioned in the target tissue 60a, the positioning introducer 116 is removed from the jacket 120b leaving at least a portion of the jacket 120 in the tissue pathway created during the positioning step. The jacket 120 is further configured to receive a microwave energy delivery device 370 as further described hereinbelow and illustrated in FIGS. 3A-3B.

In one embodiment, at least a portion of the jacket 120 lacks sufficient structural strength to maintain a form and/or a structure in the patient tissue 60 or in the target tissue 60a after the positioning introducer 116 is removed from the jacket 120. For example, during or after removal of the positioning introducer 116 a portion of the jacket 120 may collapse inward and/or upon itself. Collapsing of a portion of the jacket 120, such as the sheath 120b, as illustrated in FIG. 2B, may reduce or relieve vacuum created during the removal of the sheath 120b.

In another embodiment the cooling jacket is radiually flexible, (e.g. expandable in the radial direction). As such, the positioning introducer 116 of FIGS. 1A-1B may be formed as a smaller gage than the microwave energy delivery device 370 illustrated in FIGS. 3A-3B. During insertion, the positioning assembly 100 forms a smaller initial puncture site in patient tissue 60 that will typically stretch to accommodate the larger microwave energy delivery device 370 without enlarging or creating a further incision.

Elongated shaft 112 of positioning introducer 116 may provide a passageway for fluids to flow between the distal and proximal ends of the elongated shaft 112. For example, the elongated shaft 112 may form a tip vent hole 112b and a handle vent hole 112c fluidly connected by a lumen 112a. Lumen 112a provides a passageway for fluid (e.g., air, water, saline and/or blood) to flow through the positioning introducer 16 and in or out of the jacket 20 to relieve vacuum or pressure that may be created when the positioning introducer 16 is moved within the jacket 120.

In another embodiment, the outer surface of the elongated shaft 112 may form one or more channels (not explicitly shown) that extend longitudinally between the distal end and the proximal end of the elongated shaft 112. In yet another embodiment, the elongated shaft 112 of the positioning introducer 116 may be formed of a porous material that includes a structure that facilitates the flow of fluid longitudinally between the distal end and the proximal end of the elongated shaft 112.

The sharpened tip 121 may be configured to maintain a form and/or a structure after the removal of the positioning introducer 116 as illustrated in FIG. 2B.

FIG. 3A is a perspective view of the disassembled microwave energy delivery assembly 300 according to an embodiment of the present disclosure. Microwave energy delivery assembly 300 includes a microwave energy delivery device 370 and the jacket 320 of the positioning assembly 10 of FIGS. 1A-1B. The microwave energy delivery device 370 is configured to slideably engage jacket 320 and form a fluid-cooled microwave energy delivery assembly 300 as illustrated in FIG. 3B and described hereinbelow.

Microwave energy delivery device 370 includes an input section 378, a sealing section 380a and an antenna section 372. Input section 378 includes a fluid input port 378a and a power connector 378b. Fluid input port 378a connects to a suitable cooling fluid supply (not explicitly shown) configured to provide cooling fluid to an electrosurgical energy delivery device. A power connector 378b is configured to connect to a microwave energy source such as a microwave generator. Sealing section 380a of the microwave energy delivery device 370 interfaces with the sealing section 380b of the jacket 320 and is configured to form a fluid-tight seal therebetween. Antenna section 372 includes a microwave antenna 371 configured to radiate energy when provided with a microwave energy power signal. A cooling fluid exit port 374 resides in fluid communication with fluid input port 378a. More particularly, fluid supplied to the fluid input port 378a flows through one or more lumens formed within the microwave energy delivery device 370 and exits though the cooling fluid exit port 374. Tip 376 of the microwave energy delivery device 370 is configured to engage receptacle tip 320c of jacket 320.

FIG. 3B is a cross sectional view of the assembled microwave energy delivery assembly of FIG. 3A according to an embodiment of the present disclosure. Microwave energy delivery device 370 slideably engages jacket 320 such that the sealing section 380a and tip 376 of the microwave energy delivery device 370 engage the jacket sealing section 380b and receptacle tip 320c of the jacket 320, respectively, and form a fluid-tight seal therebetween.

In use, the energy delivery device assembly 300 is configured as a fluid-cooled microwave energy delivery device. As illustrated by the flow arrows 375 in FIG. 3B, fluid enters the fluid input port 378a and travels distally through the microwave energy delivery device 370 to the cooling fluid exit port 374. A fluid-tight engagement between the tip 376 and the receptacle tip 320c limits the flow of fluid distally relative to the cooling fluid exit port 374. Fluid that exits the cooling fluid exit port 374 flows proximally through a lumen 376 formed between the outer surface of the microwave energy delivery device 370 and the inner surface of the jacket 320 thereby cooling at least a portion of the sheath portion 320b of the jacket 320. Fluid exits the energy delivery device assembly 300 through the fluid outlet 320d.

The tip 376 of the microwave energy delivery device 370 and the receptacle tip 320c may be any suitable shape provided that tip 376 and receptacle tip 320c mutually engage one another.

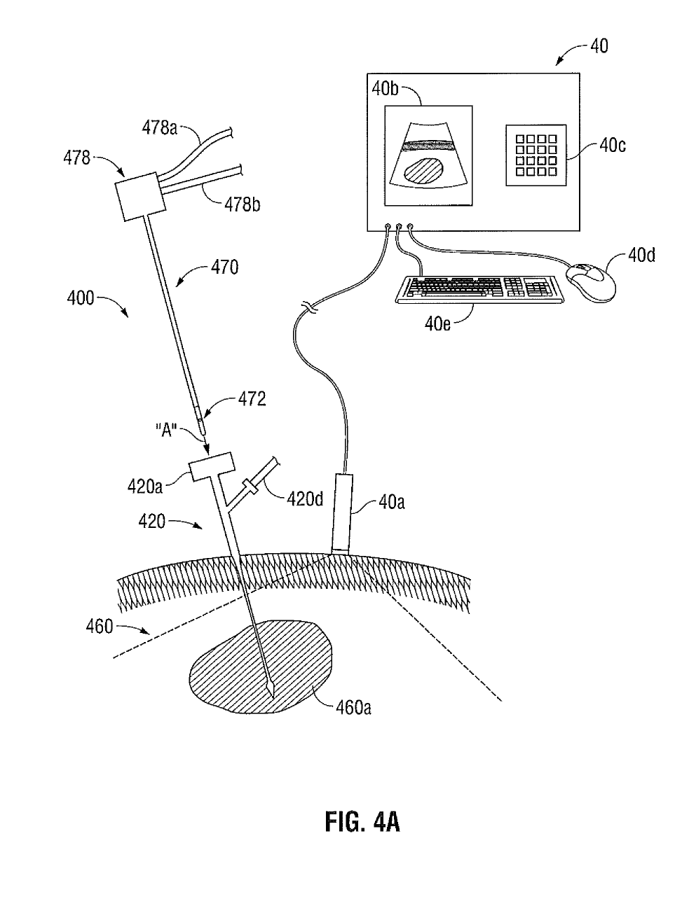

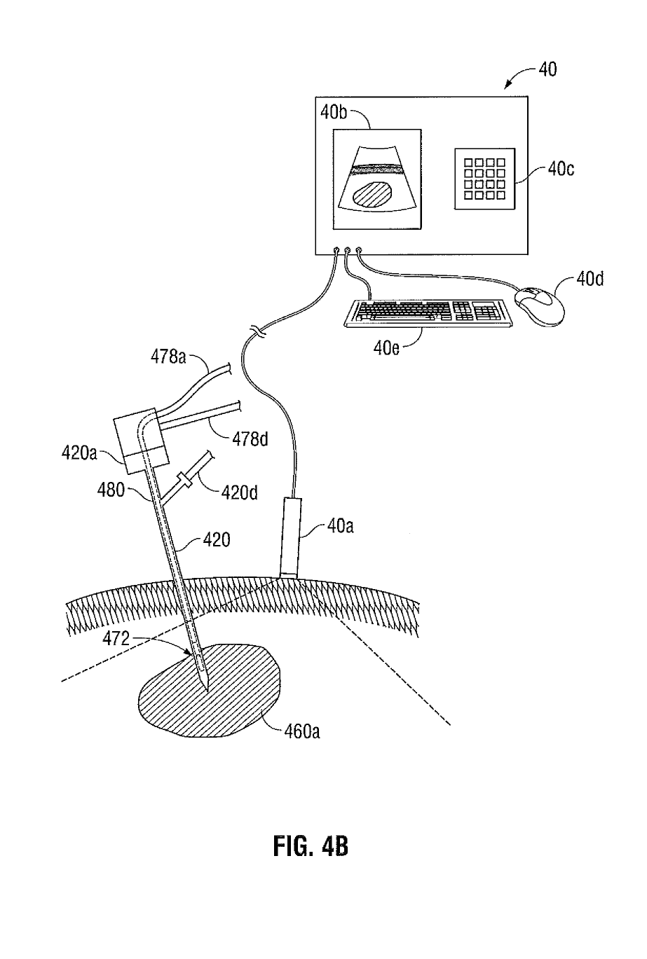

As illustrated in FIGS. 4A and 4B, an energy delivery assembly 400 includes the microwave energy delivery device 470 described similarly hereinabove and illustrated in FIGS. 3A and 3B and the jacket 420 described similarly hereinabove and illustrated in FIGS. 1A-1B and FIGS. 3A-3B and shown as 20 and 320, respectively. The jacket 420 in FIG. 4A and 4B is similar to jacket 320 of the positioning assembly 100 of FIGS. 2A-2B positioned in the pathway in tissue 460 and in the target tissue 460a.) The microwave energy delivery assembly 400 is assembled by inserting the microwave energy delivery device 470 into the jacket 420 as indicated by the arrow “A”.

After assembling the microwave energy delivery assembly 400 in the tissue pathway, a fluid supply (not shown) connects to the fluid input port 478a, a fluid drain connects to the fluid outlet 420d and a suitable microwave energy signal source connects to the power connector 478b. Fluid is circulated through the microwave energy delivery assembly 400 in a similar fashion as described above and energy is delivered to the target tissue 460a through the antenna 472 of the microwave energy delivery device 470.

After a suitable amount of energy is delivered to the target tissue 460a, the microwave energy delivery assembly 400 is removed from the tissue pathway. In one embodiment, the assembly 400 is removed by grasping the receiver portion 420a of the jacket 420 and the input section 478 of the microwave energy delivery device 470 and withdrawing the assembly from the patient.

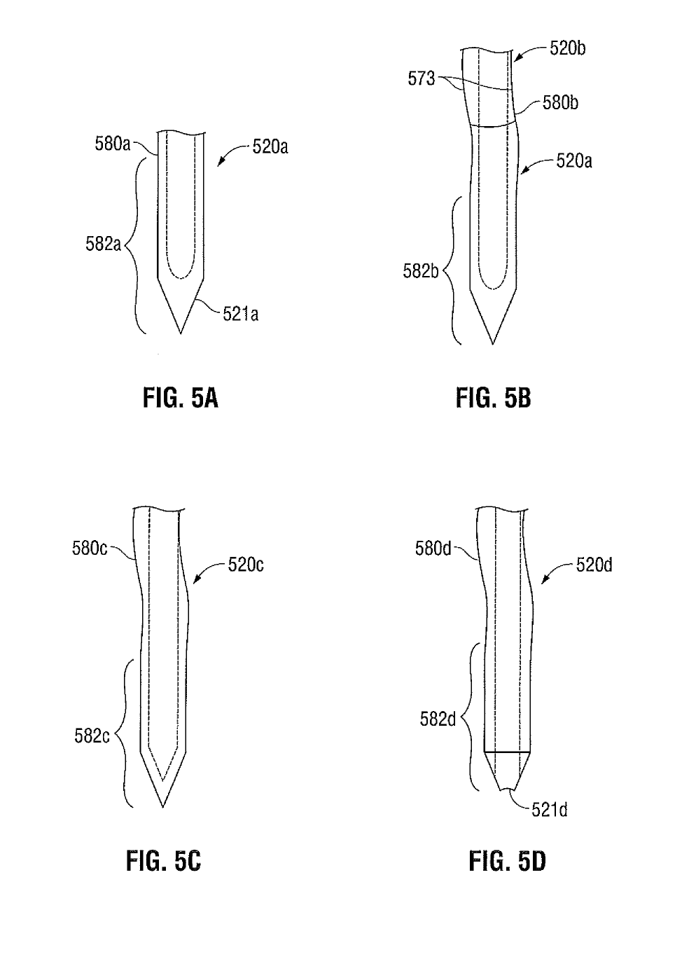

FIGS. 5A-5D are each cross-sectional views of the distal portion of a jacket 520a-520d according to various embodiments of the present disclosure. In FIG. 5A, jacket 520a includes a semi-rigid sheath 580a and a semi-rigid receptacle tip 582a. The semi-rigid receptacle tip 582a forms a sharpened tip 521a at the distal end that is sufficiently rigid to pierce tissue. In FIG. 5B, jacket 520b includes a flexible sheath 580b and a semi-rigid receptacle tip 582b. Flexible sheath 580b may stretch in diameter and/or length to accommodate the positioning introducer and/or the microwave energy delivery device when inserted into the jacket 520b as described hereinabove. In one embodiment, at least a portion of the receptacle tip 582b forms a portion of the microwave antenna 571b and radiates energy to tissue. In yet another embodiment at least a portion of the sheath 580b includes a microwave energy choke 573 capable of preventing energy from traveling proximally from the antenna.

In FIG. 5C, jacket 520c includes a flexible sheath 580c and a rigid receptacle tip 582c. Jacket 520c is configured to receive a sharpened or pointed tip. In FIG. 5D, jacket 520d includes a flexible sheath 582d and a flexible receptacle tip 582d. A distal tip 521d is configured to receive a positioning introducer and microwave energy delivery device with a sharpened tip. The receptacle tip 582d is configured to form a watertight seal between the jacket 520d and the introducer (e.g., introducer 16, see FIG. 1) and/or the delivery device (e.g., delivery device 370, see FIG. 3A) inserted therewithin.

The assemblies and methods of using the assemblies discussed above are not limited to microwave antennas used for hyperthermic, ablation, and coagulation treatments but may include any number of further microwave antenna applications. Modification of the above-described assemblies and methods for using the same, and variations of aspects of the disclosure that are obvious to those of skill in the art are intended to be within the scope of the claims.

What is claimed is:

1. An electrosurgical positioning and energy delivery system including:
a positioning introducer;
a microwave energy delivery device; and
a jacket configured to slideably receive one of the positioning introducer and the microwave energy delivery device;
wherein the positioning introducer and the jacket form a positioning assembly configured for percutaneous insertion in patient tissue, the positioning assembly configured to be visible percutaneously to an imaging system, and
wherein the microwave energy delivery device and the jacket form a microwave energy delivery assembly configured to circulate cooling fluid therethrough during delivery of microwave energy to the patient tissue.

2. The system according to claim 1, wherein the positioning introducer is hyperechoic.

3. The system according to claim 1, wherein the positioning introducer is visible to an ultrasonic imaging system.

4. The system according to claim 1, wherein the positioning introducer further includes a treatment configured to improve visibility of the positioning introducer by an ultrasonic imaging system.

5. The system according to claim 4, wherein the treatment includes a surface dispersion treatment.

6. The system according to claim 5, wherein the surface dispersion treatment is one of a dimpled surface and a surface of imbedded particles.

7. The system according to claim 4, wherein the positioning introducer further includes a resonant material that resonates when exposed to energy transmitted from the ultrasonic imaging system.

8. The system according to claim 7, wherein the resonant material is a crystalline polymer.

9. The system according to claim 4, wherein the positioning introducer further includes a geometry that resonates at the frequency of the energy transmitted from the ultrasonic imaging system.

10. The system according to claim 9, wherein the geometry is defined by at least one of wall thickness of the positioning introducer, a gap defined in a periphery of the positioning introducer, a series of grooves defined in a periphery of the positioning introducer and a fin extending from a periphery of the positioning introducer.

11. The system according to claim 1, wherein the positioning system is percutaneously visible to an MRI imaging system.

12. The system according to claim 11, wherein the positioning system further includes a non-ferromagnetic material.

13. The system according to claim 12, wherein the non-ferromagnetic material is one of a ceramic, titanium and plastic.

14. The system according to claim 1, wherein the jacket, assembled with the positioning introducer, includes a geometry at a distal end thereof to facilitate tissue penetration.

15. The system according to claim 1, wherein the microwave energy delivery assembly is adapted to connect to a microwave energy source that supplies a microwave energy signal and further is adapted to connect to a cooling fluid source that supplies cooling fluid.

16. A method for deploying an electrosurgical energy apparatus, comprising the steps of:
providing an electrosurgical positioning and energy delivery system including:
a positioning introducer;
a microwave energy delivery device; and
a jacket configured to slideably receive one of the positioning introducer and the microwave energy delivery device;
forming a positioning assembly by slideably receiving the positioning introducer within the jacket;
advancing the positioning assembly to target tissue whereby the advancement of the positioning assembly is percutaneously observed on an image system, the positioning assembly defining a pathway during tissue penetration;
withdrawing the positioning introducer from the jacket, with the jacket remaining in situ;
forming a microwave energy delivery assembly by slideably receiving the microwave energy delivery device within the jacket;
treating target tissue with electrosurgical microwave energy; and
withdrawing the microwave energy delivery assembly from the pathway.

17. The method of claim 16 further including the steps of:
connecting a fluid supply to the microwave energy delivery device and a cooling fluid return to the jacket; and
circulating the cooling fluid through at least a portion of the microwave energy delivery assembly to absorb thermal energy therefrom.
